SEQUENCE LISTING (13 sequences)

SEQ ID NO: 1 - DNA, 109 bp, Artificial sequence
  sequence encoding the EGT-FLAG epitope fusion
atgactattctctgctggcttgcactgctgtctacgcttactgctgttaacgcggactacaaggacgatgatgacaaagccatggctgctcggtaccctgcacgagctc

SEQ ID NO: 2 - DNA, 34 bp, Artificial sequence
  PCR primer
taagaagcttgccaccatggaggggaaaaatctg

SEQ ID NO: 3 - DNA, 39 bp, Artificial sequence
  PCR primer
taacggtaccgcggccgcctaaggggaatgaattttccg

SEQ ID NO: 4 - DNA, 39 bp, Artificial sequence
  PCR primer
caataagcttccatggctatgtatttgtgtctcagcaac

SEQ ID NO: 5 - DNA, 39 bp, Artificial sequence
  PCR primer
taacggtaccgcggccgcttaagccactgatttagagtg

SEQ ID NO: 6 - DNA, 60 bp, Artificial sequence
  PCR primer
ttacgatatcagatctgccaccatgtacccctacgacgtccctgactacgccatggcccg

SEQ ID NO: 7 - DNA, 34 bp, Artificial sequence
  PCR primer
ctataagctttcacagcaggttgatctcgtccag

SEQ ID NO: 8 - DNA, 70 bp, Artificial sequence
  PCR primer
ttacgatatcagatctgccaccatgtacccctacgacgtccctgactacgccatggggtgtttgggcaac

SEQ ID NO: 9 - DNA, 34 bp, Artificial sequence
  PCR primer
ctataagctttcacaagagttcgtactgcttgag

SEQ ID NO: 10 - protein, 10 aa, Artificial sequence
  conserved unit at the beginning of transmembrane domain II of olfactory receptors
PMYLFLGNLS

SEQ ID NO: 11 - protein, 10 aa, Artificial sequence
  conserved unit at the end of transmembrane domain IV and at the beginning of intracellular loop i2 of olfactory receptors
MAYDRYVAIC

SEQ ID NO: 12 - protein, 6 aa, Artificial sequence
  conserved unit at the beginning of transmembrane domain VI of olfactory receptors
FSCSSH

SEQ ID NO: 13 - protein, 6 aa, Artificial sequence
  conserved unit in transmembrane domain VII of olfactory receptors
PMLNPF

(19) United States
(12) Patent Application Publication
(10) Pub. No.: US 2006/0009618 A1
(43) Pub. Date: Jan. 12, 2006

(54) Expression receptors with 7 transmembrane domains in baculovirus-insect cell system

(76) Inventors: Gerard Devauchelle, Saint-Christol-Lez-Ales (FR); Jacques Demaille, Montferrier-Sur-Lez (FR); Conception Ferraz, Montpellier (FR); Valery Matarazzo, Bassens (FR); Catherine Ronin, Sausset-Les-Pins (FR); Martine Cerutti, Saint-Christol-Lez-Ales (FR)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/522,124

(22) PCT Filed: Jul. 22, 2003
(86) PCT No.: PCT/FR03/02309

§ 371 Date: Jul. 28, 2005

(30) Foreign Application Priority Data
Jul. 24, 2002 (FR) 02 09377

(51) Int. Cl. C07K 14/435; C12P 21/06

(52) U.S. Cl. 530/350; 435/69.1; 435/456; 435/348; 536/23.5

(57) ABSTRACT

The invention concerns the expression in a baculovirus-insect cell system of receptors with seven transmembrane domains, in particular receptors coupled to proteins (;, in particular olfactory receptors, under the transcriptional control of a promoter derived from the polyhedrin promoter by deletion of all or part of the region of said promoter extending from −1 to −12 positions relative to the initiation site of the polyhedrin translation.

The invention relates to the production, in a baculovirus-insect cell system, of receptors with seven transmembrane domains, and in particular of G-protein-coupled receptors.

The superfamily of G-protein-coupled receptors (GPCRs) with seven transmembrane domains comprises in particular many membrane receptors of neurotransmitters, of neuropeptides and of hormones. They all have the same structural organization, namely a single polypeptide chain comprising seven hydrophobic domains which cross the membrane lipid bilayer.

Among the G-protein-coupled receptors, olfactory receptors (ORs) represent a very large family and constitute, in vertebrates, the family that is numerically the largest (ZOZUYLA et al., Genome Biology, 6(2), 2001: http://www.genomebiology.com/2001/2/6/research/0018.1; ZHANG and FIRESTEIN, Nat. Neurosci. 5(2), 124-133, 2002). They are mainly located at the surface of the membranes of the neuroreceptor cells (olfactory neurons) of the olfactory epithelium, but their expression in other tissues has also been reported. A very large number of genes encoding olfactory receptors have been identified, making it possible to estimate the number of different types of olfactory receptors in a mammal at several hundred, or even several thousand. Since the initial isolation of cDNA encoding ORs from rat olfactory epithelium (BUCK and AXEL, Cell, 65 (1), 175-187, 1991), genes encoding putative ORs have been cloned from many vertebrate species, including humans, and invertebrate species (for review, see MOMBAERTS, Annual Review of Neuroscience, 22, 487-509, 1999). The total or partial sequences of these genes are accessible on the databases, and in particular are grouped together in the ORDB base accessible at the address http://ycmi.med.yale.edu/senselab/ordb/ (SKOUFOS et al., Nucl. Acids Res., 28, 341-343, 2000).

The criteria, established according to the available sequence data, which are normally used to classify a receptor with seven transmembrane domains among the olfactory receptors are as follows:

the size of the molecule: they are small receptors (approximately 300-400 amino acids), in which the intracellular and extracellular loops and the terminal segment are very short (approximately 15 to 40 amino acids for the intracellular and extracellular loops, and approximately 20 to 30 amino acids for the N-terminal segments);

the amino acid sequence homology: certain amino acid units are very conserved between the various olfactory receptors (BUCK and AXEL, Cell, 65 (1), 175-187, 1991; PILPEL and LANCET, Protein Science, 8 (5), 969-977, 1999). These are in particular units: PMYLFLGNLS (SEQ ID NO.: 10) at the beginning of transmembrane domain II, MAYDRYVAIC (SEQ ID NO.: 11) at the end of transmembrane domain IV and at the beginning of intracellular loop i2, SY at the end of transmembrane domain V, FSCSSH (SEQ ID NO.: 12) at the beginning of transmembrane domain VI and PMLNPF (SEQ ID NO.: 13) in transmembrane domain VII. Generally, the coexistence of these units is sufficient to classify a vertebrate sequence with seven transmembrane domains in the OR family. More generally, the greatest conservation between the various olfactory receptors is located in transmembrane domains II, VI and VII and intracellular loop i2. On the other hand, transmembrane domains III, IV and V are hypervariable regions.

The binding of an olfactory molecule to an olfactory receptor causes the activation of G proteins which stimulate the enzymatic cascade resulting in the production of cyclic AMP (cAMP). The cAMP in turn induces the opening of sodium and calcium ion channels, resulting in depolarization of the olfactory neuron membrane and thus inducing a nerve influx which transmits the signal to the olfactory bulb.

A second mechanism of olfactory transduction has also been proposed, involving inositol triphosphate (IP₃) instead of cAMP as second messenger (BOEKHOFF et al., EMBO Journal, 9 (8), 2453-2458, 1990; SCHILD et al., Journal of Neurophysiology, 73 (2), 862-866, 1995). Recent experiments indicate, however, that, physiologically, olfactory transduction is mainly generated by the cAMP pathway and not by that of IP₃ (BELLUSCIO et al., Neuron., 20 (1), 69-81, 1998; WONG et al., Neuron., 27 (3), 487-497, 2000; BRUNET et al., Neuron., 17 (4), 681-693, 1996).

The G proteins which have been identified as being involved in olfactory signal transduction via the cAMP pathway are Gαs and Gαolf. In the case of transduction via the IP₃ pathway, it has been reported that the Go or Gq proteins (FADOOL et al., Chemical Senses, 20, 489-498, 1995; SCHANDAR et al., J. Biol. Chem., 273, 16669-16677, 1998) could be involved.

Expression in a heterologous system potentially constitutes a major tool for the functional characterization and bioengineering of G-protein-coupled receptors.

G-protein-coupled receptors have been expressed in functional form in various heterologous systems: mention will in particular be made of the baculovirus-insect cell system, which has been used, for example, to express adrenergic receptors, muscarinic receptors, serotonin receptors, cannabinoid receptors, ocytocin receptors, substance P receptors, etc. (PARKER et al., J. Biol. Chem., 266 (1), 519-527, 1991; RICHARDSON and HOSEY, J. Biol. Chem., 267 (31), 11149-22255, 1992; VASUDEVAN et al., FEBS Lett., 311 (1), 7-11, 1992; BUTKERAIT et al., J. Biol. Chem., 270 (31) 18691-18699, 1995; NOWELL et al., Biochemical Pharmacology, 55 (11), 1893-1905, 1998; GIMPL et al., Biochemistry, 34 (42), 13794-13801, 1995; NISHIMURA et al., Journal of Receptor & Signal Transduction Research, 18 (1), 51-65, 1998), and also to reconstitute GPCR/G-protein coupling by coexpression, under the control of the polyhedrin promoter, of a receptor and of a G protein (BUTKERAIT et al., J. Biol. Chem., 270 (31), 1-8691-18699, 1995; BARR et al., J. Biol. Chem., 272 (4), 2223-2229-1997).

Expression in a heterologous system is of most particular advantage in the case of olfactory receptors, which are mainly orphelin receptors for which the ligands are not known.

Some olfactory receptors have thus been expressed in mammalian or insect cells. In the case of insect cells, RAMING et al. (Nature, 361 (3410), 353-356, 1993) have expressed the rat OR5 receptor in Sf9 cells after infection with a recombinant baculovirus, and have observed transient increases in the IP₃ second messenger in the infected cells in response to stimulation with lyral or lilial. Similar results have been reported by BREER et al. (Annals of the New York Academy of Sciences, 855, 175-181, 1998).

However, a limitation to the development of the use of heterologous expression systems, in particular in the case of olfactory receptors, comes from a relatively low level of expression of functional receptors at the cell surface. It is assumed that this problem results from poor targeting to the plasma membrane when these receptors are expressed in cells other than mature olfactory neurons. It appears that intramolecular interactions in the 3rd intracellular loop could be the cause of these proteins being retained in the intracellular compartments (GIMELBRANT et al., Journal of Neurochemistry, 72 (6), 2301-2311, 1999).

In order to improve the membrane-targeting of olfactory receptors in mammalian cells, it has been proposed to fuse them with a heterologous signal peptide. Libraries of chimeric ORs, fused with the signal peptide of a receptor for rhodopsin or for serotonin, have been expressed in HEK293 cells (KRAUTWURST et al., Cell, 95 (7), 917-926, 1998; WETZEL et al., Journal of Neuroscience, 19 (17), 7426-7433, 1999). It has thus been observed that exposure of these transfected cells to olfactory mixtures results in transient increases in intracellular calcium, and that this response is amplified when the ORs are coexpressed with the G_{α15-16} protein (also called G_{α16}), a subtype of universal G_{q} (KRAUTWURST et al., mentioned above).

It has also been proposed to express olfactory receptors in mature olfactory neurons, in order, firstly, to allow their correct targeting and insertion in the plasma membrane and, secondly, to provide a system of suitable second messengers capable of generating a signal that is sufficiently large to be detected (U.S. Pat. No. 5 993 778).

The inventors have now developed a system that improves the expression of G-protein-coupled receptors, and in particular of olfactory receptors, in insect cells.

They have put forward the hypothesis that the targeting of these receptors to the membrane could be more effective if they are expressed under the control of a promoter that is weaker than the polyhedrin promoter normally used for the expression of heterologous genes in a baculovirus.

They have tested various promoters and have noted that the use of a polyhedrin promoter that is partially truncated in order to decrease its activity makes it possible to improve targeting of the receptors to the plasma membrane of the insect host cells, and to thus obtain the surface expression of functional receptors.

The subject of the present invention is also an expression cassette comprising:

a) a promoter derived from the polyhedrin promoter of a baculovirus by deletion of all or part of the region of said promoter extending from positions −1 to −12 relative to the polyhedrin translation initiation site;

b) a sequence encoding a receptor with seven transmembrane domains, placed under the transcriptional control of said promoter.

Advantageously, in order to obtain the optimum level of expression in the context of the implementation of the present invention, use is made of a promoter in which the deleted portion comprises at least the region extending from positions −1 to −5 relative to the polyhedrin translation initiation site.

According to a preferred embodiment of the present invention, said expression cassettes also comprises, upstream of the sequence b), a sequence c) encoding a signal peptide.

By way of nonlimiting examples of sequences encoding signal peptides that can be used for implementing the present invention, mention will be made of the signal peptide of ecdysteroid UDP glucosal transferase (EGT) from the AcNPV baculovirus, the signal peptide of bovine lactotransferrin, etc.

Said expression cassette may also comprise, upstream or downstream of the sequence b) encoding the receptor with seven transmembrane domains, a sequence d) encoding a tag peptide that facilitates detection of the expressed recombinant receptor. By way of nonlimiting examples of tag peptides that can be used for implementing the present invention, mention will be made of the FLAG epitope, HA epitope, etc.

An expression cassette in accordance with the invention may be constructed so as to express any receptor with seven transmembrane domains. Particularly advantageously, said receptor is an olfactory receptor.

A subject of the present invention is also a method for expressing a receptor with seven transmembrane domains in an insect cell, characterized in that said insect cell is infected with a recombinant baculovirus comprising an expression cassette as defined above. Advantageously, said expression cassette is inserted as a replacement for the native polyhedrin promoter and gene of said baculovirus.

According to a preferred embodiment of the present invention, a G protein is also expressed in the same insect cell, which G protein may be labeled at its N-terminal end with a tag peptide, for example the HA epitope, without this impairing the expression of the protein at the surface of the insect cell or its coupling with the receptor.

According to a particularly advantageous arrangement of this embodiment, said G protein is expressed under the control of the promoter of the P10 gene of a baculovirus.

The inventors have in fact noted that the use of the promoter of the P10 gene, the expression of which is a little earlier than that of the polyhedrin gene, makes it possible to express a receptor with seven transmembrane domains in a cell already containing a considerable amount of G protein, and thus to optimize the coupling between said receptor and said protein.

Coexpression of the receptor with seven transmembrane domains and of said G protein can be carried out by co-infecting an insect cell with two recombinant baculoviruses: one expressing the receptor in an expression cassette in accordance with the invention, and the other expressing said G protein under the control of the P10 protein promoter.

However, particularly advantageously, use will be made of a double-recombinant baculovirus comprising:

an expression cassette in accordance with the invention, and

a sequence encoding a G protein placed under the transcriptional control of the promoter of the P10 gene which may also comprise, upstream, a sequence encoding a tag peptide, for example the HA epitope.

A subject of the present invention is also recombinant vectors carrying at least one expression cassette in accordance with the invention as defined above.

In this context, the present invention encompasses in particular:

transfer plasmids carrying an insert comprising: an expression cassette in accordance with the invention, as defined above, and, on either side of this cassette, baculovirus sequences homologous to those of the regions flanking the polyhedrin gene in the baculovirus into which it is desired to insert the cassette;

recombinant baculoviruses containing an expression cassette in accordance with the invention; preferably, they are double-recombinant baculoviruses also comprising a sequence encoding a G protein, under the transcriptional control of the promoter of the P10 gene. These baculoviruses can in particular be obtained, conventionally, by homologous recombination between a transfer plasmid in accordance with the invention and the genome of a baculovirus. In the case of the double-recombinant baculoviruses, an additional step of homologous recombination between a transfer plasmid comprising a sequencing coding a G protein, flanked by baculovirus sequences homologous to those flanking the sequence encoding the P10 protein in the baculovirus concerned is carried out.

To implement the present invention, use may be made of the conventional tools for cloning and expressing genes of interest in a baculovirus-insect cell system, such as those described, for example, in BACULOVIRUS EXPRESSION VECTORS: A LABORATORY MANUAL (O'REILLY et al., Freeman and Cie, New York (1994)], and also in a large number of patents or patent applications, such as, for example, application EP 0 651 815, application EP 0 638 647 or PCT application WO 95/20672.

A subject of the present invention is also insect cells infected with a recombinant baculovirus in accordance with the invention; these cells express, at their surface, functional receptors with seven transmembrane domains, capable of coupling either to endogenous baculovirus G proteins, when they are expressed alone, or to exogenous G proteins coexpressed in the same cell.

The insect cells according to the present invention can be used for studying and characterizing receptors with seven transmembrane domains, in particular for verifying the functionality of putative receptors, identifying ligands for orphelin receptors, or studying mechanisms involved in the activation of the receptor, signal transduction, for example the identification of the G protein(s) which is (are) partner(s) of the receptor concerned, the step of coupling to these proteins, and the function of the complex thus formed, etc.

The signal resulting from the binding of these receptors with their ligand(s) can be detected by various techniques which are known in themselves. For example, it is possible to detect variations in intracellular calcium, in particular by calcium imaging, or variations in the second messengers, inositol phosphate or cyclic AMP; it is also possible to detect the phosphorylation and/or the kinase activity of the proteins involved in the transduction cascade, etc.

A subject of the present invention is thus in particular:

a method for identifying a functional receptor with seven transmembrane domains, and/or identifying the G protein partner of a receptor with seven transmembrane domains, characterized in that it comprises the following steps:

bringing an insect cell in accordance with the invention expressing a receptor or a receptor/G protein combination to be tested into contact with a mixture of potential ligands for said receptor;

detecting the signal resulting from the binding of said receptor with one of the ligands present in said mixture.

A method for identifying a ligand for a receptor with seven transmembrane domains, characterized in that it comprises the following steps:

bringing each ligand to be tested into contact with an insect cell in accordance with the invention, expressing a functional receptor with seven transmembrane domains, and a G protein partner of said receptor;

identifying the ligand(s) being sought, by detection of the signal resulting from its (their) binding with said receptor.

A method for identifying a receptor with seven transmembrane domains corresponding to a ligand of interest, characterized in that it comprises the following steps:

bringing said ligand into contact with an insect cell in accordance with the invention expressing a receptor with seven transmembrane domains to be tested and a G protein partner of said receptor;

identifying the receptor(s) being sought, by detection of the signal resulting from its (their) binding with said ligand of interest.

In this context, use may in particular be made of insect cells in accordance with the invention for expressing mutant receptors with seven transmembrane domains, and screening the mutant(s) that recognize(s) a ligand of interest.

Particularly advantageously, the insect cells according to the present invention are used as described above, for studying and characterizing olfactory receptors.

The olfactory receptor ligands may be olfactory molecules, olfactory binding proteins (OBPs), olfactory molecule/OBP complexes, or synthetic molecules such as cyclodextrins used in perfumery (soaps, candles, papers, etc) as olfactory fixers that very gradually release the volatile olfactory molecule.

Thus, it is possible to use insect cells in accordance with the invention, for studying the functionality of putative olfactory receptors, and/or of various receptor/G protein combinations, in particular by testing their response to mixtures of olfactory molecules, mixtures of OBPs, mixtures of olfactory molecule/OBP complexes, or mixtures of synthetic molecules as defined above.

When a functional receptor has been identified, it is possible to use insect cells in accordance with the invention, expressing said receptor and a G protein partner of said receptor, for identifying the ligand(s) for the receptor concerned, by separately testing the response of this receptor to various olfactory molecules or olfactory molecule/OBP complexes. It is also possible to use insect cells in accordance with the invention, expressing functional olfactory receptors and G protein partners of said receptors, for identifying those which respond to a ligand of interest, by separately testing the response of each of these receptors to various olfactory molecules or olfactory molecule/OBP complexes.

Insect cells in accordance with the invention, expressing a functional olfactory receptor for which the ligand has been identified, can advantageously be used for the production of biosensors, allowing the detection of olfactory molecules, in particular for the analysis and the quality control of volatile components, among which are aromatic products of interest, and/or for the detection of products that may affect organoeleptic qualities, or for the detection of potentially harmful products.

For example, the present invention can be implemented in order to:
express and purify orphelin olfactory receptors in various species (for example humans, domestic animals, etc.);
produce antibody specific for these receptors, that can be used in the immunocytochemical typing of neuroepithelium taken clinically, for example in the case of a sheathing cell transplant in damaged spinal cord;
screen olfactory molecules or OBPs (natural or recombinant) for pharmacological use;
screen functional mutant olfactory receptors or OBPs that have been mutated, using olfactory molecules of interest, for example food flavorings, scents, fragrances, etc.

The present invention will be understood more fully from the further description which follows, which refers to nonlimiting examples of expression, in accordance with the invention, of functional olfactory receptors in insect cells.

### EXAMPLE 1

### Production of Recombinant Baculoviruses Expressing Human Olfactory Receptors Under the Control of a Modified Polyhedrin Promoter

Construction of Transfer Vectors

Vector pGmAc 217:

This vector contains an insert, obtained from the EcoRI I fragment of approximately 7 kb of the AcMNPV baculovirus, comprising the polyhedrin region, by deletion of the fragment −8 to +502 (relative to the polyhedrin translation initiation site) and replacement of the deleted portion with a BglII linker. The insert thus obtained therefore comprises a polh polyhedrin promoter from which 8 pb (positions −1 to −8) have been deleted.

Insertion of a Signal Peptide and of a Labeling Sequence:

An oligonucleotide is inserted into the vector pGmAc 217, immediately downstream of the modified polh promoter. The sequence of this oligonucleotide is as follows:

(SEQ ID NO.: 1)

5′-ATGA ACT ATT CTC TGC TGG CTT GCA CTG CTG TCT ACG
CTT ACT GCT GTT AAC GCG GAC TAC AAG GAC GAT GAT
GAC AAA GCC ATG GCT GCT CGG TAC CCT GCA CGA GCT
C-3′.

This oligonucleotide comprises a 54 pb sequence (underlined) encoding the signal peptide of ecdysteroid UDP glucosyl transferase (EGT) from the AcNPV baculovirus (GenBank M22619), followed by the sequence encoding the FLAG epitope (in italics), itself followed by two unique NcoI and KpnI restriction sites (in bold).

Genes Encoding the Olfactory Receptors

The genes, without introns, encoding the putative olfactory receptors OR-209 and OR-210 from human chromosome 17 (p13.3) are amplified by PCR using cosmide No. ICRF105cF06137 (GenBank No. HSU53583).

The following sense (S) and antisense (AS) of the oligonucleotide primers are used:

OR-209 NcoI S:

(SEQ ID NO.: 2)

5′-TAA GAA GCT TGC CAC CAT GGA GGG GAA AAA TCT
G-3′;
OR-209 KpnI AS:

(SEQ ID NO.: 3)

5′-TAA CGG TAC CGC GGC CGC CTA AGG GGA ATG AAT TTT
CCG-3′;
OR-210 NcoI S:

(SEQ ID NO.: 4)

5′-CAA TAA GCT TCC ATG GCT ATG TAT TTG TGT CTC AGC
AAC-3′;
OR-210 KpnI AS:

(SEQ ID NO.: 5)

5′-TAA CGG TAC CGC GGC CGC TTA AGC CAC TGA TTT AGA
GTG-3′.

The amplifications are carried out in the following reaction mixture:

10 ng of the cosmide DNA preparation, 10 mM of Tris-HCl (pH 8.3), 50 mM of KCl, 2.5 mM of MgCl₂, 0.001% of gelatine, 0.2 mM of dNTP, 1.5 U of Expand™ polymerase mixture (ROCHE MOLECULAR BIOCHEMICALS) and 100 pmol of each of the primers; and according to the following program: 94° C. (90 sec, 1 cycle), 94° C. (20 sec), 50° C. (25 sec) and 72° C. (90 sec) (40 cycles); 72° C. (120 sec, 1 cycle).

The two PCR products are digested with NcoI/KpnI and the resulting fragments are subcloned between the NcoI and KpnI sites of the vector pGmAc 217 containing the signal peptide.

The transfer vectors obtained, called pGmAc 217-209 and pGmAc 217-210 contain, respectively, the sequence encoding the OR-209 receptor and the sequence encoding the OR-210 receptor.

Construction of the Recombinant Baculoviruses

The recombinant viruses are obtained by cotransfection of Sf9 cells with the DNA of the wild-type baculovirus AcMNPV and the DNA of the transfer vector pGmAc 217-209 or pGmAc 217-210.

The spodoptera frugiperda Sf9 cells (ATCCCRL 1711) are cultured in 25 cm² or 75 cm² plastic flasks and maintained at 28° C. in a TC100 medium (GIBCO-BRL) containing 5% of fetal bovine serum (GIBCO-BRL). Subculturing is performed twice a week.

The wild-type AcMNPV baculovirus DNA is prepared from viral particles.

The cotransfection is carried out by lipofection, by incubating 2×10⁶ cells with 3 ml of serum-free medium containing 500 ng of baculovirus DNA and 10 μg a of transfer vector, mixed with 40 μl of DOTAP (ROCHE, France). After incubation at 28° C. for 4 hours, the transfection mixture is removed and replaced with 5 ml of medium supplemented with serum. The recombinant baculoviruses are recovered from the supernatant of the transfected cells after incubation for 5 days at 28° C.

The recombinant baculoviruses are isolated by means of three lysis plaque purification steps and are selected on the basis of an ob-phenotype (absence of inclusion bodies), and then multiplied by means of successive cell infections. The recombinant baculoviruses, hereinafter referred to as AcMNPV209 and AcMNPV210, contain, respectively, the sequence encoding the OR-209 receptor and the sequence encoding the OR-210 receptor.

The recombinant baculoviruses are stored at 4° C. until they are used.

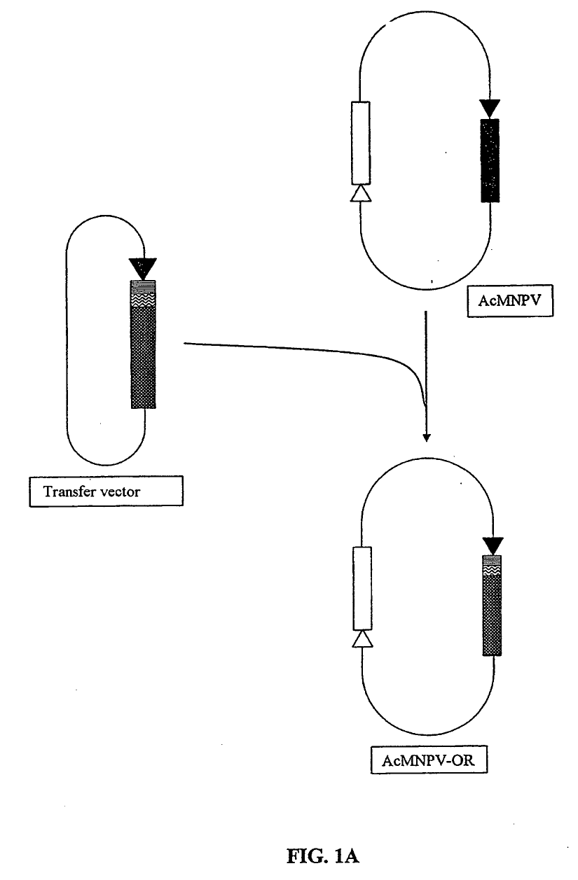

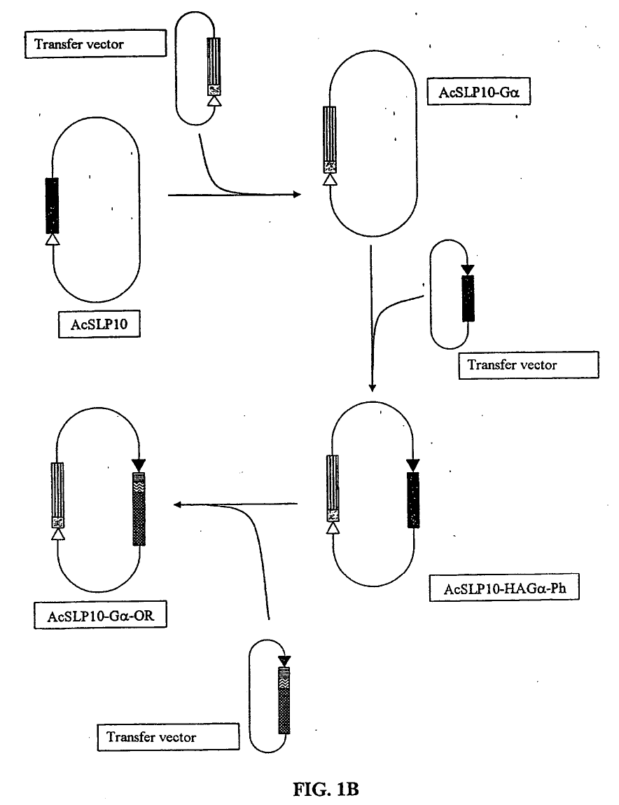

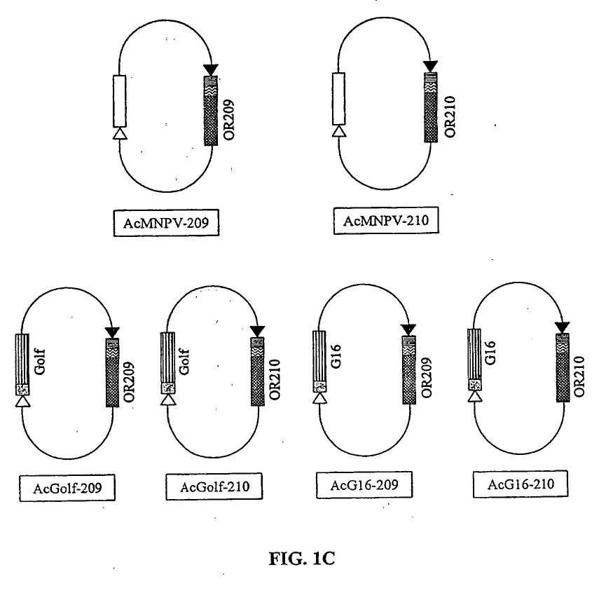

FIG. 1A represents a general diagram of the various steps for constructing a recombinant baculovirus comprising a sequence encoding an OR receptor, combined with the signal peptide of the EGT gene and with the FLAG epitope labeling sequence. The AcMNPV209 and AcMNPv210 baculoviruses are represented in FIG. 1C. Legend of FIG. 1:
▪ Polyhedrin
▾ Polyhedrin promoter
□ P10
Δ P10 promoter
[symbol] Olfactory receptor
[symbol] EGT signal peptide
[symbol] FLAG epitope
□ HA epitope
[symbol] G protein

### EXAMPLE 2

### Production of a Double-Recombinant Baculovirus Expressing a Human Olfactory Receptor Under the Control of a Modified Polyhedrin Promoter, and a G Protein Under the Control of the P10 Promoter

Construction of Transfer Vectors

Host Vector:

The vector p119 (LEMEULLE et al., FEBS Lett., 423, 159-166, 1998) containing the unique Bg1II and HindIII cloning sites located downstream of the baculovirus p10 promoter is used.

Genes Encoding G Proteins

Gα₁₆ Protein

The cDNA encoding the Ga16 protein was obtained from a cDNA library of the human promyelocytic leukemia line HL60 (CLONTECH, France). The HA-epitope labeling sequence is introduced by PCR.

The sense (S) and antisense (AS) oligonucleotides which were used are as follows:

G_{α16} Bg1II HA S:

(SEQ ID NO.: 6)

5′-TTA CGA TAT CAG ATC TGC CAC CAT GTA CCC CTA CGA
CCT CCC TGA CTA CGC CAT GGC CCG CTC GCT GAC C-3′;
G_{α16} HindIII AS:

(SEQ ID NO.: 7)

5′-CTA TAA GCT TTC ACA GCA GGT TGA TCT CGT CCA
G-3′.

The amplification is carried out in the following reaction mixture:

10 ng of cDNA preparation, 10 mM of Tris-HCl (pH 8.3), 50 mM of KCl, 2.5 mM of MgCl₂, 0.001% of gelatine, 0.2 mM of dNTP, 1.5 U of Expand™ polymerase mixture (ROCHE MOLECULAR BIOCHEMICALS) and 100 pmol of each of the primers;

and according to the following program: 94° C. (60 sec, 1 cycle), 94° C. (45 sec), 58° C. (45 sec) and 72° C. (90 sec) (35 cycles); 72° C. (120 sec, 1 cycle).

G_{αolf} Protein

The cDNA encoding the G_{αolf} protein was cloned by RT-PCR from a whole brain total RNA preparation provided by Dr. Gilles TOUMANIANTZ (IPMC, Nice, France). The mRNAs are obtained by incubating (5 min at 70° C. and 5 min at 4° C.) 4 μg of total RNA with 1 μg of oligonucleotide dT primer (INVITROGEN) in a final volume of 15 μl. 10 μl of the mRNA preparation are then used as a sample for the reverse transcription into cDNA using the “cDNA cycle” kit from INVITROGEN (The Netherlands). The reverse transcription is carried out at 42° C. for 70 min and the reaction is inactivated at 94° C. for 5 min. The synthesized cDNA is used as a matrix for the subsequent PCR amplifications.

The HA epitope labeling sequence is introduced by PCR.

The sense (S) and antisense (AS) oligonucleotides used for this PCR are as follows:

G_{αolf} Bg1II HA S:

(SEQ ID NO.: 8)

5′-TTA CGA TAT CAG ATC TGC CAC CAT GTA CCC CTA CGA
CGT CCC TGA CTA CGC CAT GGG GTG TTT GGG CAA C-3′;
G_{αolf} HindIII AS:

(SEQ ID NO.: 9)

5′-CTA TAA GCT TTC ACA AGA GTT CGT ACT GCT TGA
G-3′.

The PCR amplification conditions are identical to those used for G_{α16}.

The two PCR products are digested with BglII/HindIII and the resulting fragments are subcloned between the BglII and HindIII sites of the vector p119.

The transfer vectors obtained, called p119-HA-G_{α16} and p119-HA-G_{αolf} pGmAc 217-209, contain, respectively, the sequence encoding HA-G_{α16} and the sequence encoding HA-G_{αolf}.

Construction of the Recombinant Baculoviruses

Recombinant Baculoviruses Expressing the G_{α16} Protein or the G_{αolf} Protein Under the Control of the Promoter of the P10 Gene

Spodoptera frugiperda Sf9 cells cultured as described in example 1 above are cotransfected with the AcSLP10 baculovirus DNA and one of the vectors p119-HA-G_{α16} or p119-HA-G_{αolf}.

The AcSLP10 baculovirus (CHAABIHI et al., J. Virol., 67, 2664-2671, 1993) is a modified baculovirus which has a single strong late promoter (P10), which controls the expression of the sequence encoding polyhedrin.

The cotransfection is carried out by lipofection, as described in example 1 above. The recombinant baculoviruses obtained, called AcSLP10G₁₆ and AcSLP10G_{olf}, express, respectively, p119-HA-G₁₆ or p119-HA-G_{olf}, under the control of the p10 promoter. These baculoviruses are multiplied as described in example 1 above.

Double-Recombinant Baculoviruses

The Spodoptera frugiperda Sf9 cells are cotransfected with the AcSLP10 baculovirus DNA and one of the vectors p119-HA-G₁₆ or p119-HA-G_{olf}, and the transfer vector pGmAcI50 (DEVAUCHELLE and CERUTTI, Les Baculovirus d'Insectes: Vecteurs d'expression de genes etrangers [Insect baculoviruses: Expression vectors for foreign genes], Regard sur la Biochimie [Biochemistry overview], No. 5, C2, 1991) which contains the sequence encoding polyhedrin under the control of its own promoter. The recombinant baculoviruses expressing the G proteins and the polyhedrin are selected on the basis of their ob+ phenotype (presence of inclusion bodies), and are multiplied. The recombinant baculoviruses obtained, expressing p119-HA-G₁₆ or p119-HA-G_{olf}, respectively, under the control of the p10 promoter, are called AcSLP10G₁₆Ph and AcSLP10G_{olf}Ph.

The DNA of each of the AcSLP10G₁₆Ph or AcSLP10G_{olf}Ph baculoviruses is used to cotransfect Sf9 cells with the DNA of the pGmAc 217-209 baculovirus or of the pGmAc 217-210 baculovirus. The double-recombinant baculoviruses obtained are selected on the basis of an ob-viral phenotype. These double-recombinant baculoviruses are referred to as AcG₁₆-209; AcG_{olf}-210; AcG₁₆-209; AcG_{olf}-210.

FIG. 1B represents a general diagram of the various steps for constructing a double-recombinant baculovirus comprising a sequence encoding a G protein combined with the HA epitope-labeling sequence under the control of the P10 promoter, and a sequence encoding an OR receptor, combined with the signal peptide of the EGT gene and with the FLAG epitope-labeling sequence, under the control of the modified promoter of the polyhedrin gene. The AcG₁₆-209, AcG_{olf}-210, AcG₁₆-209 and AcG_{olf}-210 baculoviruses are represented in FIG. 1C.

### EXAMPLE 3

### Expression of Olfactory Receptors and of G Proteins in Insect Cells

Sf9 cells are harvested 36-48 hours after infection. The production of olfactory receptors or of recombinant G proteins is evaluated by immunoelectrophoretic blotting, using antibodies directed against their FLAG or HA epitope tag.

The cells infected with the AcMNPV209 and AcMNPV210 baculoviruses express proteins recognized by the anti-FLAG antibody, and having a molecular weight corresponding to that of the ORs.

The cells infected with the AcSLP10G₁₆ and AcSLP10G_{olf} baculoviruses express proteins recognized by the anti-HA antibody, and having a molecular weight corresponding to that of the G proteins.

The cells infected with the double-recombinant baculoviruses AcG₁₆-209, AcG_{olf}-210, AcG₁₆-209 and AcG_{olf}-210 express the 2 types of proteins.

The cellular location of the ORs and of the G proteins were studied by immunofluorescence 36 hours after infection. The ORs are detected by means of an anti-FLAG primary antibody and an FITC-labeled secondary antibody. The G proteins are detected by means of a rhodamine-labeled anti-HA antibody.

The labeled cells are observed by confocal microscopy. In the cells infected with the AcMNPV209 and AcMNPV210 baculoviruses, labeling is observed to be located at the cell surface, with a punctate distribution typical of membrane receptors. In the cells infected with the AcSLP10G₁₆ and AcSLP10G_{olf} baculoviruses, labeling is observed to have a submembrane cytoplasmic location. In the cells infected with the double-recombinant baculoviruses AcG₁₆-209, AcG_{olf}-210, AcG₁₆-209 and AcG_{olf}-210, colocalization of the receptor with each G protein co-expressed is observed. No major difference is observed between OR17-209 and OR17-210 as regards preferential binding with G_{olf} or G₁₆. It therefore appears that these two receptors are capable of coupling to the two G proteins in insect cells.

### EXAMPLE 4

### Functionality of the Receptors Expressed in the Insect Cells

In a first series of experiments, the possibility of measuring the calcium response in Sf9 cells using the calcium imaging technique was tested.

The protocol used for the assays is as follows:

Sf9 cells are cultured on 24-well plates containing 12 mm coverslips. Before the experiments, the culture medium is removed and washed with a buffer containing 10 mM of NaCl, 60 mM of KCl, 25 mM of MgCl₂, 1.8 mM of CaCl₂, 4 mM of D-glucose, 110 mM of sucrose and 10 mM of 2-(N-morpholino)ethanesulfonic acid; the pH is adjusted to 6.2 at ambient temperature with Trizma base.

The cells are incubated for 1 hour at ambient temperature in the dark with a solution of MBS containing fura 2-AM (4 μm) (molecular probe). The cells are then washed with an excess of MBS without fura 2-AM and mounted in a Coverwell chamber (Polylabo, France) (diameter: 9 mm; thickness: 2.5 mm; cross section: 22.5).

The chamber is placed under the microscope and the cells are perfused under gravity with 0.85 ml/min (3.78 cm/min) at ambient temperature (23° C.).

The fura-2 fluorescence measurements are carried out using a T.I.L.L Photonics GmbH, (Planegg) Polychrome II multipath wavelength illumination system for extinction. The microscope (Olympus) is equipped with a long-distance objective (×20). The spatio-temporal Ca²⁺ distributions are studied using a PCO interline camera. The excitation wavelength of the dye varies alternately between 340 and 380 nm and is adjusted to the exposure time of the camera (20-40 msec; binning: 2). The acquisition and the calculation of the fluorescence images are carried out using the T.I.L.L-Vision program. All the signals are related to the background noise. The fluorescence ratios (f₃₄₀/f₃₈₀) are calculated for the intracellular Ca²⁺ variations.

The effect of the following products on intracellular Ca²⁺ variation was tested:

the ionophore 4-bromo-A23187, used at a concentration of 5 μM;

octopamine, used at a concentration of 50 μM, as a control for the ability of the Sf9 cells to increase the intracellular calcium concentration in response to activation of the octopamine receptor, which is a G protein/IP3-coupled receptor naturally present in Sf9 cells;

the HENKEL 100 mixture (HENKELL), which is a mixture of 100 different aroma compounds at the same concentration (1% w/w). This solution is diluted to 1/10 000 in the culture medium before use.

All the products are added to the perfusion by means of a manual distributor.

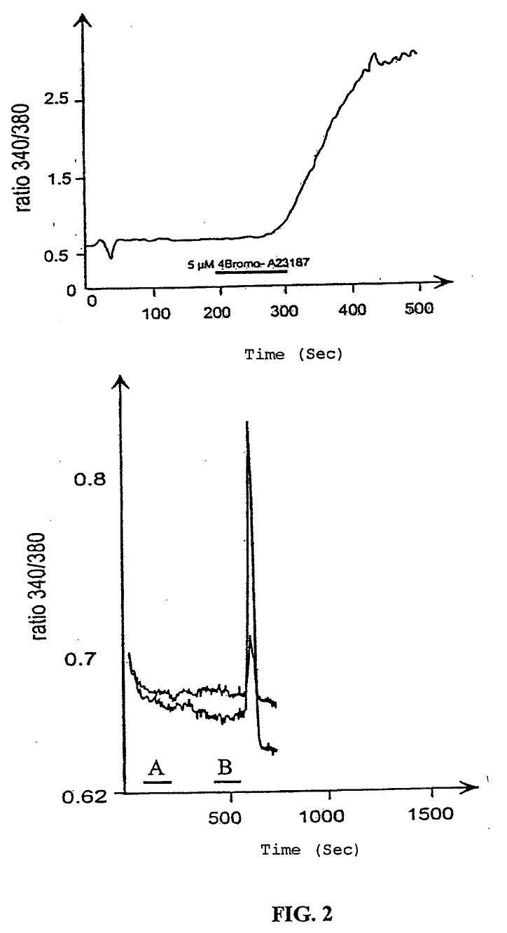

The results obtained on non-infected Sf9 cells are given in FIG. 2. The Ca²⁺ signals are the result of the mean determined on all the cells in the field of the camera (gray signal) and of the mean for the responding cells (black signal) (n=4). It is observed that the ionophore 4-bromo A23187 induces a large increase in intracellular calcium (top graph). On the other hand, the HENKEL 100 olfactory mixture (bottom graph, A) does not produce any change in intracellular calcium, whereas octopamine (bottom graph, B) induces an increase in intracellular calcium. These results show that Sf9 cells do not possess endogenous olfactory receptors whose activity could interfere with that of recombinant receptors expressed in these cells.

In a second series of experiments, the ability of human OR17-209 and OR17-210 receptors, alone or co-expressed with G proteins, to respond to the olfactory molecules was tested. Sf9 cells are infected with the AcMNPV, AcMNPV209, AcMNPV210, AcG₁₆-209, AcG₁₆-210, AcG_{olf}-209 or AcG_{olf}-210 baculoviruses and, 36 hours after infection, the cellular responses to the HENKEL 100 mixture (10 μM) or to octopamine (50 μM) are recorded by calcium imaging, as described above for the non-infected cells.

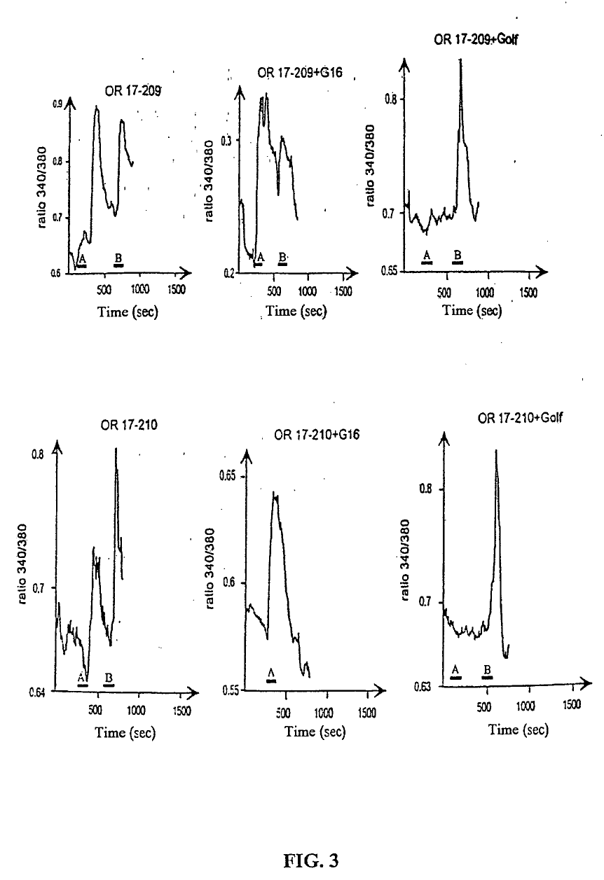

The results are given in FIG. 3. The Ca²⁺ signals are the result of the mean from the responding cells within the field of the camera [receptor OR17-209 (n=12)/OR17-210 (n=6)/OR17-210-G₁₆ (n=10)/OR17-209-G₁₆ (n=15)/OR17-209-G_{olf} (n=6)/OR17-210-G_{olf} (n=4)].

A: stimulation with the HENKEL 100 mixture;

B: stimulation with octopamine.

These results show that the HENKEL 100 olfactory mixture induces a transient [Ca²⁺]ᵢ signal for the two receptors OR17-209 and OR17-210 expressed alone or co-expressed with the G₁₆ protein. On the other hand, no increase in intracellular calcium is observed in the case of the receptors expressed with the G_{olf} protein. In all these cells, octopamine induces an increase in intracellular calcium.

Although co-expression of the ORs in Sf9 cells with the Ga16 protein conserves the calcium response, their co-expression with the G_{olf} proteins results in a disappearance of this response. This demonstrates that, in insect cells, the response to the olfactory molecules is based on the release of Ca²⁺ due to the coupling of the olfactory receptors to endogenous G proteins.

The above results clearly show that the human olfactory receptors OR17-209 and OR17-210 can be functionally expressed in Sf9 cells, alone or with the G_{α16} protein.

When the olfactory receptors are expressed with the G_{αolf} protein, no change in [Ca²⁺]ᵢ is observed subsequent to the application of olfactory molecules. This could be explained by the fact that the expression of G_{αolf} in Sf9 cells diverts the signaling cascade to a pathway which becomes undetectable by calcium imaging. In olfactory neurons, it is known that the binding of olfactory molecules to olfactory receptors brings about, via the G_{αolf} protein, the production of cAMP which directly opens cyclic nucleotide-gated (CNG) calcium channels in the plasma membrane. This type of channel is probably absent in Sf9 insect cells, which results in it being impossible to detect a calcium response when the olfactory receptors are co-expressed with the G_{αolf} protein in these Sf9 cells. These results also show that the olfactory receptors co-expressed with the G_{αolf} protein preferably couple with this protein rather than with the endogeneous G proteins.

1. An expression cassette comprising:
a) a promoter derived from the polyhedrin promoter of a baculovirus by deletion of all or part of the region of said promoter extending from positions −1 to −12 relative to the polyhedrin translation initiation site;
b) a sequence encoding a receptor with seven transmembrane domains, placed under the transcriptional control of said promoter.

2. The expression cassette as claimed in claim 1, characterized in that it also comprises, upstream of the sequence b), a sequence encoding a signal peptide.

3. The expression cassette as claimed in claim 1, characterized in that said receptor with seven transmembrane domains is an olfactory receptor.

4. A method for expressing a receptor with seven transmembrane domains in an insect cell, characterized in that said insect cell is infected with a recombinant baculovirus comprising an expression cassette as claimed in claim 1.

5. The method as claimed in claim 4, characterized in that a G protein is also expressed in the same insect cell.

6. The method as claimed in claim 5, characterized in that said G protein is expressed under the control of the promoter of the P10 gene of a baculovirus.

7. The method as claimed in claim 6, characterized in that use is made of a double-recombinant baculovirus comprising:
an expression cassette comprising:
a) a promoter derived from the polyhedrin promoter of a baculovirus by deletion of all or part of the region of said promoter extending from positions −1 to −12 relative to the polyhedrin translation initiation site;
b) a sequence encoding a receptor with seven transmembrane domains, placed under the transcriptional control of said promoter; and
a sequence encoding a G protein placed under the transcriptional control of the promoter of the P10 gene of said baculovirus.

8. A recombinant baculovirus comprising an expression cassette as claimed in claim 1.

9. The recombinant baculovirus as claimed in claim 8, characterized in that said expression cassette is inserted as a replacement for the polyhedrin promoter and gene of said baculovirus.

10. The recombinant baculovirus as claimed in claim 9, characterized in that said baculovirus also comprises a sequence encoding a G protein placed under the transcriptional control of the promoter of the P10 gene.

11. An insect cell infected with a recombinant baculovirus as claimed in claim 8.

12. The use of an insect cell as claimed in claim 11, for determining the functionality of a putative receptor with seven transmembrane domains.

13. The use of an insect cell as claimed in claim 11, for identifying the ligand(s) for an orphelin receptor with seven transmembrane domains.

14. The use of an insect cell as claimed in claim 11, for identifying (a) receptor(s) with seven transmembrane domains capable of binding to a ligand of interest.

15. The use as claimed in claim 12, characterized in that said receptor with seven transmembrane domains is an olfactory receptor.

16. The expression cassette as claimed in claim 2, characterized in that said receptor with seven transmembrane domains is an olfactory receptor.

17. A recombinant baculovirus comprising an expression cassette as claimed in claim 2.

18. A recombinant baculovirus comprising an expression cassette as claimed in claim 16.
